(19) United States
(12) Patent Application Publication
(10) Pub. No.: US 2020/0306162 A1
(43) Pub. Date: Oct. 1, 2020

(54) COSMETIC MINERAL SUNSCREEN COMPOSITIONS FOR SKIN

(71) Applicant: L'OREAL, Paris (FR)

(72) Inventor: Rabia AHMAD, Queens, NY (US)

(73) Assignee: L'OREAL, Paris (FR)

(21) Appl. No.: 16/367,499

(22) Filed: Mar. 28, 2019

(51) Int. Cl. A61K 8/58; A61K 8/49; A61K 8/06; A61K 8/891; A61K 8/81; A61K 8/29; A61K 8/27; A61K 8/19; A61K 8/28; A61K 8/26; A61Q 17/04

(52) U.S. Cl. CPC A61K 8/585; A61K 8/4946; A61K 8/06; A61K 8/891; A61K 8/8152; A61Q 17/04; A61K 8/27; A61K 8/19; A61K 8/28; A61K 8/26; A61K 8/29

(57) ABSTRACT

The present disclosure relates to anhydrous mineral sunscreen cosmetic composition comprising: a) From about 0.1% to about 2% of one silica silylate; b) From about 0.1% to about 5% of allantoin; c) From about 1% to about 12% of one at least one emulsifier; d) From about 0.1 to about 5% of at least one film former; e) From about 1% to about 25% of one or more mineral UV filtering agents; f) From about 20% to about 30% by weight of at least a first silicone having a viscosity from about between 2 cst to about 70 cst; and g) From about 40% to about 95% by weight of at least a second silicone; wherein all weight percentages are based on the total weight of the cosmetic composition.

### FIELD OF THE DISCLOSURE

The present disclosure relates to a cosmetic mineral sunscreen composition. More specifically, the present disclosure is directed to anhydrous cosmetic mineral sunscreen compositions containing allantoin and mineral UV filtering agents, providing a skin barrier protection from UV radiations.

### BACKGROUND

A variety of compositions, especially cosmetic compositions, have been developed to provide occlusive, transformative skin texture. Unfortunately, many of these compositions are in fact difficult to apply and do not possess a smooth feel upon application.

Although glycerin is a fairly low cost humectant or hydrating agent, problems arise when incorporating high levels of glycerin in cosmetic compositions. Incorporating high levels of glycerin results in a cosmetic compositions having a tacky and sticky feel upon application to skin. The tacky or oily feel is undesirable to consumers. Several approaches, such as using light emollients, powders, or combinations thereof may reduce tackiness; however, the resulting cosmetic compositions may not provide sufficient consumer appeal and may still have residual tackiness that can be felt on the skin after application.

It is an object of the present disclosure to provide an anhydrous cosmetic mineral sunscreen composition that provides high glide and skin protection with an elegant feel. Yet another object of the present disclosure is to provide a favorable environment for skin healing/repair/skin protection, and associated with mineral UV filtering agents sun filters.

### SUMMARY OF THE DISCLOSURE

The instant disclosure relates to sunscreen compositions that provide a surprisingly high skin barrier protection and are aesthetically pleasing. The sunscreen compositions include mineral UV filtering agents, which are known to be non-irritating, and gentle on the skin. One drawback with mineral-based sunscreen compositions is that they often appear white when applied to the skin. Consumers prefer sunscreen compositions to appear natural (unnoticeable). Developing mineral-based sunscreen products having a high Sun Protection Factor (SPF) that exhibit minimal or no whitening, however, is challenging.

The instant disclosure relates to an anhydrous mineral sunscreen cosmetic composition comprising:
a) From about 0.1% to about 2% by weight of one silica silylate;
b) From about 0.1% to about 5% by weight of allantoin;
c) From about 1% to about 30% by weight of at least one emulsifier;
d) From about 0.1 to about 5% by weight of at least one film former;
e) From about 1% to about 25% by weight of one or more mineral UV filtering agents;
f) From about 20% to about 30% by weight of at least a first silicone having a viscosity from about between 2 cst to about 70 cst; and
g) From about 40% to about 95% by weight of at least a second silicone; wherein all weight percentages are based on the total weight of the cosmetic composition.

In one or more embodiments, the silica silylate is silica silylate particles and especially aerogel particles of hydrophobic silica surface-modified with trimethylsilyl groups. In some embodiments, the silica silylate is present in an amount from about 0.5% to about 1.5% by weight based on the total weight of the composition.

In one or more embodiments, the allantoin is present in an amount from about 0.1% to about 5% by weight based on the total weight of the composition. In more embodiments, the allantoin is present in an amount from about 0.4% to about 3% by weight based on the total weight of the composition.

In some embodiments, the at least one emulsifier comprises an organosiloxane emulsifier. In one or more embodiments, the organosiloxane emulsifier is a crosslinked organosiloxane emulsifier selected from the group consisting of dimethicone crosspolymer, dimethicone/dimethicone crosspolymer, dimethicone/dimethicone PEG/PPG 15 crosspolymer, dimethicone PEG-10 crosspolymer, dimethicone PEG-10/15 crosspolymer, dimethicone PEG-15 crosspolymer, dimethicone polyglycerin-3 crosspolymer, dimethicone PPG-20 crosspolymer, dimethiconol/methylsilanol/silicate crosspolymer; dimethiconol/silicate crosspolymer, lauryl dimethicone PEG-15 crosspolymer, lauryl dimethicone polyglycerin-3 crosspolymer, PEG-8 dimethicone polysorbate-20 crosspolymer, PEG-10 dimethicone/vinyl dimethicone crosspolymer, PEG-10 lauryl dimethicone crosspolymer, PEG-15/lauryl dimethicone crosspolymer, PEG-15 laurylpolydimethylsiloxyethyl crosspolymer.

In one embodiment, the at least one film former is selected from the group consisted of acrylates/dimethicone crosspolymer, Trimethylsiloxysilicate (and) polypropylsilsesquioxane, Acrylates/Polytrimethylsiloxymethacrylate Copolymer, Dimethicone (and) Acrylates/dimethicone copolymer, Polypropylsilsesquioxane, Trimethylsiloxysilicate), and a mixture thereof.

In some embodiments, the one or more mineral UV filtering agents comprises titanium dioxide, zinc oxide, iron oxides, cerium oxides, zirconium oxides, alumina, aluminum hydroxide, or a mixture thereof. In one or more embodiments, the one or more mineral UV filtering agents may be titanium dioxide, zinc oxide, or a mixture thereof. In some embodiments, the one or more mineral UV filtering agents are present in an amount from about 3% to about 20% by weight based on the total weight of the composition.

In one embodiment, the at least first silicone is having a viscosity from about between 4 cst to about 65 cst by weight based on the total weight of the composition.

In one embodiment, the weight ratio of the emulsifier with the first silicone is about 0.4.

In one or more embodiments, the at least second silicone is selected from the group consisting of dimethicone, cyclomethicone, polysilicone-11, phenyl trimethicone, trimethylsilylamodimethicone, and stearoxytrimethylsilane. In one or more embodiments, the at least second silicone has a viscosity of at least about 100 cSt. In more embodiments, the at least second silicone comprises dimethicone. In some embodiments, the at least second silicone is present from about 43% to about 90% of silicone by weight based on the total weight of the composition.

In some embodiments, the anhydrous cosmetic composition may further comprise one or more active agents. In some embodiments, the one or more active agents are selected from the group consisting of adenosine, 2-[4-(2-hydroxyethyl)piperazin-1-yl]ethanesulfonic acid (HEPES), hyaluronic acid, lanolin, citric acid, malic acid, lactic acid, tartaric acid, salicylic acid, vitamin C, a vitamin, a retinoid, retinal, retinoic acid, a carotenoid, an amino acid, a protein, an enzyme, and a coenzyme.

Another aspect of the instant disclosure can include an anhydrous mineral sunscreen cosmetic composition comprising:
a) From about 0.1% to about 2% by weight of one silica silylate;
b) From about 0.1% to about 5% by weight of allantoin;
c) From about 1% to about 30% by weight of dimethicone crosspolymer;
d) From about 0.1 to about 2% by weight of at least one film former selected from the group consisted of acrylates/dimethicone crosspolymer, Trimethylsiloxysilicate (and) polypropylsilsesquioxane, Acrylates/Polytrimethylsiloxymethacrylate Copolymer, Dimethicone (and) Acrylates/dimethicone copolymer, Polypropylsilsesquioxane and mixture thereof.
e) From about 3% to about 20% of mineral UV filtering agents sun filters by weight based on the total weight of the composition;
f) From about 20% to about 30% by weight of at least a first silicone having a viscosity from about between 2 cst to about 70 cst; and
g) From about 40% to about 95% by weight of at least a second silicone; wherein all weight percentages are based on the total weight of the cosmetic composition.

Another aspect of the anhydrous mineral sunscreen cosmetic composition is that the composition exhibits an SPF of at least 15. In one or more embodiments, the anhydrous mineral sunscreen cosmetic composition exhibits an SPF of at least 20. In one embodiment, the anhydrous mineral sunscreen cosmetic composition exhibits an SPF of at least 30.

Another aspect of the present disclosure is a method for protecting skin from UV radiation comprising applying an effective amount of the sunscreen composition of the present disclosure to the skin.

The anhydrous mineral sunscreen cosmetic compositions of the instant disclosure provide an unexpected and unique texture that help to restore skin smoothness, reduce visual redness, improve skin tone evenness, flatten imperfections and improve skin firmness that can be spread on the skin very easily with a matte finish. They also provide protection against UV radiations.

It is to be understood that both the foregoing general description and the following detailed description are exemplary and explanatory only, and are not restrictive of the invention.

### DETAILED DESCRIPTION OF THE DISCLOSURE

The instant disclosure relates to anhydrous mineral sunscreen compositions.

The anhydrous mineral sunscreen compositions, in their broadest sense, typically include the following:
a) From about 0.1% to about 2% by weight of one silica silylate;
b) From about 0.1% to about 5% by weight of allantoin;
c) From about 1% to about 30% by weight of at least one emulsifier;
d) From about 0.1 to about 5% by weight of at least one film former;
e) From about 1% to about 25% by weight of one or more mineral UV filtering agents;
f) From about 20% to about 30% by weight of at least a first silicone having a viscosity from about between 2 cst to about 70 cst; and
g) From about 40% to about 95% by weight of at least a second silicone; wherein all weight percentages are based on the total weight of the cosmetic composition.

The anhydrous mineral sunscreen compositions of the instant disclosure exhibit a surprisingly skin protection without feeling tacky or having a high shine. The compositions are particularly interesting in that they are helping to restore skin smoothness, reduce visual redness, improve skin tone evenness, flatten imperfections and improve skin firmness. The compositions spread easily, do not irritate skin through friction and do not feel tacky even though there is a high level of silicone. Furthermore, they include mineral UV filtering agents and still keep all the properties cited above without showing any whitening.

As used herein, the term “anhydrous” means that no water is added to the composition and water is contained only in the form of the constitutional water which in some cases cannot be avoided and is brought in as part of the ingredients in very small amounts.

In some embodiments, the anhydrous cosmetic composition is essentially free of water. In one or more embodiments, the amount of water may be less than 0.5%. In some embodiments, the amount of water may be less than 0.1%. In one or more embodiments, the anhydrous cosmetic composition is free of water.

The term “mineral UV filtering agent” is interchangeable with the terms “mineral UV screening agent,” “inorganic UV filtering agent,” “inorganic UV screening agent,” “mineral UV filter, and “inorganic UV filter.” Mineral UV filtering agents are compounds which do not include any carbon atoms in their chemical structures that are capable of screening out or absorbing UV radiation between 280 and 400 nm.

As used herein, the expression “at least one” is interchangeable with the expression “one or more” and thus includes individual components as well as mixtures/combinations.

The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

The term “INCI” is an abbreviation of International Nomenclature of Cosmetic Ingredients, which is a system of names provided by the International Nomenclature Committee of the Personal Care Products Council to describe personal care ingredients.

In some embodiments, the anhydrous cosmetic composition is essentially free of fatty substances comprising vegetable or plant oils. In one or more embodiments, the amount of fatty substances comprising vegetable or plant oils may be less than 2% by weight. In some embodiments, the amount of fatty substances comprising vegetable or plant oils may be less than 1.8% by weight. In some embodiments, the amount of fatty substances comprising vegetable or plant oils may be less than 1.6% by weight. In some embodiments, the amount of fatty substances comprising vegetable or plant oils may be less than 1.4% by weight. In some embodiments, the amount of fatty substances comprising vegetable or plant oils may be less than 1.2% by weight. In one or more embodiments, the anhydrous cosmetic composition is free of fatty substances comprising vegetable or plant oils.

### Silica Aerogel

According to the present disclosure, the anhydrous cosmetic composition may comprise at least one silica aerogel. Silica aerogels are porous materials obtained by replacing (by drying) the liquid component of a silica gel with air. They are generally synthesized via a sol-gel process in a liquid medium and then dried, usually by extraction with a supercritical fluid, the one most commonly used being supercritical CO₂. Drying of this type makes it possible to avoid contraction of the pores and of the material. The sol-gel process and the various drying operations are described in detail in Brinker C. J. and Scherer G. W., Sol-Gel Science, New York, Academic Press, 1990.

The hydrophobic silica aerogel particles used in the present invention have a specific surface area per unit of mass (SM) ranging from 500 to 1500 m²/g, preferably from 600 to 1200 m²/g and better still from 600 to 800 m²/g, and a size expressed as the mean volume diameter (D[0.5]), ranging from 1 to 30 μm, preferably from 5 to 25 m, better still from 5 to 20 μm and even better still from 5 to 15 μm.

The specific surface area per unit of mass may be determined via the BET (Brunauer-Emmett-Teller) nitrogen absorption method described in the Journal of the American Chemical Society, vol. 60, page 309, February 1938 and corresponding to the international standard ISO 5794/1 (appendix D). The BET specific surface area corresponds to the total specific surface area of the particles under consideration.

The size of the silica aerogel particles may be measured by static light scattering using a commercial granulometer such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is especially described in the publication by Van de Hulst, H. C., “Light Scattering by Small Particles,” Chapters 9 and 10, Wiley, New York, 1957.

The silica aerogel particles used in the present invention may advantageously have a tamped density r) ranging from 0.04 g/cm³ to 0.10 g/cm³ and preferably from 0.05 g/cm³ to 0.08 g/cm³.

In the context of the present disclosure, this density, known as the tamped density, may be assessed according to the following protocol:

40 g of powder are poured into a measuring cylinder; the measuring cylinder is then placed on a Stav 2003 machine from Stampf Volumeter; the measuring cylinder is then subjected to a series of 2500 packing motions (this operation is repeated until the difference in volume between two consecutive tests is less than 2%); the final volume Vf of packed powder is then measured directly on the measuring cylinder. The tamped density is determined by the ratio m/Vf, in this instance 40/Vf (Vf being expressed in cm³ and m in g).

According to one embodiment, the hydrophobic silica aerogel particles used in the present disclosure have a specific surface area per unit of volume S_{V} ranging from 5 to 60 m²/cm³, preferably from 10 to 50 m²/cm³ and better still from 15 to 40 m²/cm³.

The specific surface area per unit of volume is given by the relationship:

S_{V}=S_{M}.r where r is the tamped density expressed in g/cm³ and SM is the specific surface area per unit of mass expressed in m²/g, as defined above.

Preferably, the hydrophobic silica aerogel particles according to the disclosure have an oil-absorbing capacity, measured at the wet point, ranging from 5 to 18 mL/g, preferably from 6 to 15 mL/g and better still from 8 to 12 mL/g.

The oil-absorbing capacity measured at the wet point, noted Wp, corresponds to the amount of water that needs to be added to 100 g of particle in order to obtain a homogeneous paste.

It is measured according to the wet point method or the method for determining the oil uptake of a powder described in standard NF T 30-022. It corresponds to the amount of oil adsorbed onto the available surface of the powder and/or absorbed by the powder by measuring the wet point, described below:

An amount m=2 g of powder is placed on a glass plate, and the oil (isononyl isononanoate) is then added dropwise. After addition of 4 to 5 drops of oil to the powder, mixing is performed using a spatula, and addition of oil is continued until a conglomerate of oil and powder has formed. At this point, the oil is added one drop at a time and the mixture is then triturated with the spatula. The addition of oil is stopped when a firm, smooth paste is obtained. This paste must be able to be spread on the glass plate without cracking or forming lumps. The volume Vs (expressed in mL) of oil used is then noted. The oil uptake corresponds to the ratio Vs/m.

The aerogels used according to the present disclosure are aerogels of hydrophobic silica, preferably of silylated silica (INCI name: silica silylate).

The term “hydrophobic silica” means any silica whose surface is treated with silylating agents, for example halogenated silanes such as alkylchlorosilanes, siloxanes, in particular dimethylsiloxanes such as hexamethyldisiloxane, or silazanes, so as to functionalize the OH groups with silyl groups Si—Rn, for example trimethylsilyl groups.

As regards the preparation of hydrophobic silica aerogel particles surface-modified by silylation, reference may be made to U.S. Pat. No. 7,470,725.

Use will be made in particular of silylated silica aerogel particles and especially aerogel particles of hydrophobic silica surface-modified with trimethylsilyl groups (trimethylsiloxyl silica).

As hydrophobic silica aerogels that may be used in the disclosure, examples that may be mentioned include the aerogel sold under the name VM-2260 (INCI name: Silica silylate), by the company Dow Corning, the particles of which have an average size of about 1000 microns and a specific surface area per unit mass ranging from 600 to 800 m²/g.

In other embodiments, the aerogels sold by the company Cabot under the names Aerogel TLD 201®, Aerogel OGD 201®, and Aerogel TLD 203®, CAB-O-SIL TS-530, CAB-O-SIL TS-610, CAB-O-SIL TS-720, Enova Aerogel MT 1100®, and Enova Aerogel MT 1200®, may be chosen.

Use will be made more particularly of the aerogel sold under the name VM-2270 (INCI name: Silica silylate) by the company Dow Corning, the particles of which have a mean size ranging from 5-15 microns and a specific surface area per unit mass ranging from 600 to 800 m²/g. It has an oil absorption capability of 1090 mL/100 g based on isononyl isononanoate.

The silica silyliate may be present in an amount from about 0.1%, 0.2%, 0.3%, 0.4%, 0.5%, 0.6%, 0.7%, 0.8%, 0.9% to about 0.9%, 1%, 1.2%, 1.4%, 1.6%, 1.8%, or to 2% by weight based on the total weight of the composition.

### Allantoin

Examples of suitable active ingredient in solid form which may be mentioned include, but are not limited to, vitamin C, alpha hydroxy acids, calamine, allantoin, refreshing active agents such as menthol, proteins and protein hydrolysates, sugar and derivatives thereof, glycyrrhetinic acid, and UV blockers. Preferred among the active ingredients in solid form is allantoin.

The active ingredient will typically be present in the composition in an amount from 0.01%, 0.02%, 0.03%, 0.04%, 0.05%, 0.1%, 0.15%, 0.2%, 0.25%, 0.3%, 0.35%, 0.4%, 0.5%, 0.6%, 0.7%, 0.8%, 0.9%, 1%, 1.5% to about 1.5%, 2%, 2.5%, 3%, 3.5%, 4%, 4.5%, or 5% by weight based on the total weight of the composition.

### Emulsifiers

Suitable examples of emulsifiers include polyether substituted linear or branched polysiloxane copolymers. For example, the emulsifier can be selected from dimethicone crosspolymer, dimethicone/vinyl dimethicone crosspolymer, PEG-10 dimethicone, PEG-9 polydimethylsiloxyethyl dimethicone and PEG-9, lauryl PEG-9 polydimethylsiloxyethyl dimethicone, dimethicone, dimethicone(and)dimethicone/PEG-10/15 crosspolymer PEG/PPG-18/18 dimethicone, dimethicone/dimethicone crosspolymer, dimethicone(and)dimethicone/polyglycerin-3 crosspolymer and combinations thereof. One preferred co-emulsifier is PEG-10 dimethicone available under the tradename of ES-5612 from Dow Corning Corporation (Midland, Mich.), or KF-6017 from Shin-Etsu (Akron, Ohio). One preferred emulsifier is polydimethyl siloxane available under the tradename of Dowsil EI-9241 DM Silicone Elastomer Blend; Dow Corning EI-9241 Dm Silicone Elastomer Blend from Dow Corning Corporation (Midland, Mich.). Another preferred emulsifier is dimethicone (and) PEG/PPG-18/18 dimethicone available under the tradename of ES-5226 DM from Dow Corning Corporation (Midland, Mich.). other suitable emulsifiers include, PEG-9 polydimethylsiloxyethyl dimethicone available under the tradename KF-6028 and PEG-9, lauryl PEG-9 polydimethylsiloxyethyl dimethicone available under the tradename KF-6038, both available from Shin-Etsu (Akron, Ohio).

The at least one emulsifier may be present in an amount from about 1%, 2%, 3%, 4%, 5%, 6%, 6.5%, 7%, 7.5%, 8%, 8.5%, 9%, 9.5%, 10%, 10.5%, 11%, 12% to about 13%, 14%, 15%, 16%, 17%, 18%, 19%, 20%, 22%, 24%, 25%, 28%, or 30% by weight based on the total weight of the composition.

### Film Former

The anhydrous cosmetic composition according to the disclosure, may include at least one film former. In some embodiments, suitable example of film formers includes, but are not limited to, Trimethylsiloxysilicate (and) polypropylsilsesquioxane, Acrylates/Polytrimethylsiloxymethacrylate Copolymer, Dimethicone (and) Acrylates/dimethicone copolymer, Polypropylsilsesquioxane, Trimethylsiloxysilicate), Crotonic acid/vinyl C8-12, Isoalkyl Esters/Va/Bis-Vinyldimethicone Crosspolymer.

The film former may be present in an amount from about 0.01%, 0.02%, 0.05%, 0.08%, 0.1%, 0.2% 0.25%, 0.3%, 0.35%, 0.4%, 0.5%, 0.6%, 0.7%, 0.8%, 0.9%, 1%, 1.5% to about 1%, 1.5%, 2%, 2.5%, 3%, 3.5%, or 4% by weight based on the total weight of the composition.

Mineral UV filtering agents

Non-limiting examples of mineral UV filtering agent include treated or untreated metal oxides such as, for example, pigments or nanopigments of titanium oxide (amorphous or crystallized in rutile and/or anatase form), of iron oxide, of zinc oxide, of zirconium oxide or of cerium oxide. Particularly preferred mineral UV filtering agents include titanium dioxide and/or zinc oxide.

In some instances, the mean particle size may be about 5 nm to about 25 μm, about 10 nm to about 10 μm, or about 15 nm to about 5 μm. The mineral UV filtering agents may be nano-pigments having a mean particle size of about 5 nm to about 100 nm, about 5 nm to about 75 nm, or about 10 nm to 50 nm. Larger particles sizes may also be useful, for example about 1 μm to about 25 μm, about 5 μm to about 20 μm, or about 10 μm to about 15 μm.

Treated pigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminium salts of fatty acids, metal (titanium or aluminium) alkoxides, polyethylene, silicones, proteins (collagen or elastin), alkanolamines, silicon oxides, metal oxides, sodium hexametaphosphate, alumina or glycerol.

The treated pigments may be titanium oxides treated with:
silica and alumina, such as the products “Microtitanium Dioxide MT 500 SA” and “Microtitanium Dioxide MT 100 SA” from the company Tayca, and the products “Tioveil Fin”, “Tioveil OP”, “Tioveil MOTG” and “Tioveil IPM” from the company Tioxide;
alumina and aluminium stearate, such as the product “Microtitanium Dioxide MT 100 T” from the company Tayca;
alumina and aluminium laurate, such as the product “Microtitanium Dioxide MT 100 S” from the company Tayca;
iron oxides and iron stearate, such as the product “Microtitanium Dioxide MT 100 F” from the company Tayca;
silica, alumina and silicone, such as the products “Microtitanium Dioxide MT 100 SAS”, “Microtitanium Dioxide MT 600 SAS” and “Microtitanium Dioxide MT 500 SAS” from the company Tayca;
sodium hexametaphosphate, such as the product “Microtitanium Dioxide MT 150 W” from the company Tayca;
octyltrimethoxysilane, such as the product “T-805” from the company Degussa;
alumina and stearic acid, such as the product “UVT-M160” from the company Kemira;
alumina and glycerol, such as the product “UVT-M212” from the company Kemira;
alumina and silicone, such as the product “UVT-M262” from the company Kemira.

Other titanium oxide pigments treated with a silicone are TiO₂ treated with octyltrimethylsilane and for which the mean size of the elementary particles is between 25 and 40 nm, such as the product sold under the trade name “T805” by the company Degussa Silices, TiO₂ treated with a polydimethylsiloxane and for which the mean size of the elementary particles is 21 nm, such as the product sold under the trade name “70250 Cardre UF TiO2SI3” by the company Cardre, anatase/rutile TiO₂ treated with a polydimethylhydrogenosiloxane and for which the mean size of the elementary particles is 25 nm, such as the product sold under the trade name “Microtitanium Dioxide USP Grade Hydrophobic” by the company Color Techniques.

Uncoated titanium oxide pigments are sold, for example, by the company Tayca under the trade names “Microtitanium Dioxide MT 500 B” or “Microtitanium Dioxide MT 600 B”, by the company Degussa under the name “P 25”, by the company Wackher under the name “Oxyde de titane transparent PW”, by the company Myoshi Kasei under the name “UFTR”, by the company Tomen under the name “ITS” and by the company Tioxide under the name “Tioveil AQ”.

The uncoated zinc oxide pigments are, for example:
those sold under the name “Z-Cote” by the company Sunsmart;
those sold under the name “Nanox” by the company Elementis; and
those sold under the name “Nanogard WCD 2025” by the company Nanophase Technologies.

The coated zinc oxide pigments are, for example:
those sold under the name “Zinc Oxide CS-5” by the company Toshibi (ZnO coated with polymethylhydrogenosiloxane);
those sold under the name “Nanogard Zinc Oxide FN” by the company Nanophase Technologies (as a 40% dispersion in Finsolv TN, C12-C15 alkyl benzoate);
those sold under the name “Daitopersion ZN-30” and “Daitopersion ZN-50” by the company Daito (dispersions in cyclopolymethylsiloxane/oxyethylenated polydimethylsiloxane, containing 30% or 50% of nanozinc oxides coated with silica and polymethylhydrogenosiloxane);
those sold under the name “NFD Ultrafine ZNO” by the company Daikin (ZnO coated with perfluoroalkyl phosphate and copolymer based on perfluoroalkylethyl as a dispersion in cyclopentasiloxane);
those sold under the name “SPD-Z1” by the company Shin-Etsu (ZnO coated with silicone-grafted acrylic polymer, dispersed in cyclodimethylsiloxane);
those sold under the name “Escalol Z100” by the company ISP (alumina-treated ZnO dispersed in an ethylhexyl methoxycinnamate/PVP-hexadecene/methicone copolymer mixture);
those sold under the name “Fuji ZNO-SMS-10” by the company Fuji Pigment (ZnO coated with silica and polymethylsilsesquioxane); and
those sold under the name “Nanox Gel TN” by the company Elementis (ZnO dispersed at a concentration of 55% in C12-C15 alkyl benzoate with hydroxystearic acid polycondensate).

The uncoated cerium oxide pigments are sold under the name “Colloidal Cerium Oxide” by the company Rhone-Poulenc. The uncoated iron oxide nanopigments are sold, for example, by the company Arnaud under the names “Nanogard WCD 2002 (FE 45B)”, “Nanogard Iron FE 45 BL AQ”, “Nanogard FE 45R AQ” and “Nanogard WCD 2006 (FE 45R)” or by the company Mitsubishi under the name “TY-220”. The coated iron oxide nanopigments are sold, for example, by the company Arnaud under the names “Nanogard WCD 2008 (FE 45B FN)”, “Nanogard WCD 2009 (FE 45B 556)”, “Nanogard FE 45 BL 345” and “Nanogard FE 45 BL” or by the company BASF under the name “Transparent Iron Oxide”.

Mixtures of metal oxides may also be used, especially of titanium dioxide and of cerium dioxide, including the silica-coated equal-weight mixture of titanium dioxide and of cerium dioxide, sold by the company Ikeda under the name “Sunveil A”, and also the alumina, silica and silicone-coated mixture of titanium dioxide and of zinc dioxide, such as the product “M 261” sold by the company Kemira, or the alumina, silica and glycerol-coated mixture of titanium dioxide and of zinc dioxide, such as the product “M 211” sold by the company Kemira.

The total amount of mineral UV filtering agents in the mineral sunscreen compositions can vary but is typically about 1 to about 40 wt. %, based on the total weight of the sunscreen composition. In some instances, the total amount of mineral UV filtering agents may be about 1 to about 30 wt. %, about 1 to about 25 wt. %, about 1 to about 20 wt. %, about 1 to about 15 wt. %, about 1 to about 10 wt. %, about 5 to about 40 wt. %, about 5 to about 30 wt. %, about 5 to about 25 wt. %, about 5 to about 20 wt. %, or about 5 to about 10 wt. %, based on the total weight of the sunscreen composition.

### Silicones

Useful silicones include, but are not limited to, polyorganosiloxanes, polyalkylsiloxanes, polyarylsiloxanes, polyalkarylsiloxanes, polyestersiloxanes, and a mixture thereof. Non-limiting examples include dimethicone, cyclomethicone (cyclopentasiloxane), amodimethicone, trimethyl silyl amodimethicone, phenyl trimethicone, trimethyl siloxy silicate, polymethylsilsesquioxane and a mixture thereof.

In some instances, the compositions include one or more silicones selected from the group consisting of polydimethylsiloxanes (dimethicones), polydiethylsiloxanes, polydimethyl siloxanes having terminal hydroxyl groups (dimethiconols), polymethylphenylsiloxanes, phenylmethylsiloxanes, amino functional polydimethylsiloxane (amodimethicone), non-ionic dimethicone copolyols, dimethicone copolyol esters, dimethicone copolyol quaternium nitrogen containing compounds, dimethicone copolyol phosphate esters, and mixtures thereof.

### First Silicone

Examples of silicones with a low viscosity and that may be mentioned include those with a viscosity from about 2 cst to about 70 cst. In some embodiments, the viscosity of the silicones might be from about 2 cst, 3 cst, 4 cst, 5 cst, 6 cst, 7 cst, 8 cst, 10 cst, 12 cst, 14 cst, 16 cst, 18 cst, 20 cst, 22, cst, 24 cst, 26 cst, 28 cst, 30 cst, 35 cst, 40 cst to about 40 cst, 42 cst, 44 cst, 46 cst, 48 cst, 50 cst, 52 cst, 54 cst, 56 cst, 58 cst, 60 cst, 62 cst, 64 cst, 66 cst, 68 cst, or 70 cst.

Non-limiting examples include Dow Corning® 200 Fluids, DC 200® fluid, DC 200 silicone, Dow Corning® Silicone, Dow Corning® Dimethicone, GE SF96 Silicone, SF96 Silicone, Wacker AK Fluids, Baysilone Dimethicones, Bayer-Baysilone Silicone, Shinetsu Silicone, Shinetzu Dimethicone, cosmetic grade silicone, personal care dimethicone

The amount of the at least first silicone may be in an amount of about 20%, 21%, 22%, 23%, 24%, 25% to about 25%, 26%, 27%, 28%, 29%, or 30% by weight based on the total weight of the composition.

### Second Silicone

Examples of silicones having a high viscosity that may be mentioned include those with a viscosity from at least about 100 cst or even more.

Non-limiting examples include dimethicone, cyclomethicone, polysilicone-11, phenyl trimethicone, trimethylsilylamodimethicone, and stearoxytrimethylsilane.

Other non-limiting examples of silicones having a high viscosity higher than that of the first silicone include those non-emusifying silicones that are available in combination with emulsifying silicones in the following combinations: Cyclopentasiloxane (and) PEG/PPG-19/19 Dimethicone, PEG/PPG-19/19 Dimethicone (and) C13-16 Isoparaffin (and) C10-13 Isoparaffin, Dimethicone (and) PEG/PPG-18 Dimethicone, Cyclopentasiloxane (and) and PEG/PPG-18/18 Dimethicone, Dimethicone (and) PEG/PPG-18/18 Dimethicone.

Non-limiting examples of silicones having a high viscosity higher than that of the first silicone are commercially available under the following tradenames: Dow Corning 200® Fluid, 100cs, Dow Corning 200®, 200cs, Dow Corning 200®350cs, Dow Corning 200®5000cs, Dow Corning 200®1000cs, GE SF96-100cs, GE SF96-200cs, GE SF 96-350, GE SF96-500, and GE SF96-1000.

In one embodiment, the second silicone is available as DOWSIL EL-9140 DM Silicone Elastomer Blend.

The amount of the at least second silicone may be in an amount of about 40%, 41%, 42%, 43%, 44%, 45%, 47% 50%, 52%, 54%, 56%, 58%, 60%, 61%, 62%, 63%, 64%, 65%, 66%, 67%, 68%, 69%, 70%, 72%, 74% to about 74%, 76%, 78%, 80%, 82%, 84%, 86%, 88%, 90%, 92%, 93%, 94% or 95% by weight based on the total weight of the composition

### Active Agents

The anhydrous mineral sunscreen cosmetic composition according to the disclosure, may further include one or more active ingredients. The cosmetic composition according to the disclosure includes an active ingredient from about 0.01% to about 5% by weight based on the total weight of the composition. In one embodiment, the active ingredient is capryloyl salicylic acid, adenosine, baicalin, resveratrol, other polyphenols, or combinations thereof. In another embodiment, the active ingredient is an organic or inorganic UV filter, or combination thereof. In some embodiments, the active ingredient is selected from humectant, antimicrobial, antioxidant, preservative, vitamin, vitamin derivative, UV filter, vegetable extract; and dye/pigment, filler, thickener, polymer, penetrant, fragrance, dispersant, film-forming agent; ceramide; opacifier and combinations thereof. In one embodiment, one or more actives can be selected from sodium hydroxide, disodium EDTA, sodium citrate, sodium hyaluronate, capryloyl salicylic acid, lactic acid, methyl dihydro jasmonate, acetyl trifluoromethyl phenyl valyglycine, pentaerythrityl tetra-di-t-butyl hydroxydrocinnamate, n-hydroxysuccinimide, palmitoyl oligopeptide, chrysin, palmitoyl tetrapeptide-7, yeast extract, citric acid and combinations thereof.

### Fragrance

Fragrance including natural or synthetic odoriferous substances or mixtures thereof may be included in the cosmetic composition of the present disclosure. Use may be made of mixtures of different odoriferous substances which together generate an attractive scent. Natural odoriferous substances are extracts of flowers (lily, lavender, rose, jasmine, neroli or ylang-ylang), stems and leaves (geranium, patchouli, petitgrain), fruits (anis, coriander, caraway, juniper), fruit rinds (bergamot, lemon, orange), roots (mace, angelica, celery, cardamom, costus, iris, thyme), needles and twigs (spruce, fir, pine, mountain pine) and resins and balsams (galbanum, elemi, benzoin, myrrh, frankincense, opoponax). Typical synthetic perfume compounds are products of the esters, ethers, aldehydes, ketones, alcohols and hydrocarbon types. Essential oils of low volatility, which are generally used as flavoring components, are also suitable as fragrances, for example, but not limited to, sage oil, camomile oil, clove oil, balm oil, peppermint oil, cinnamon leaf oil, linden blossom oil, juniper berry oil, vetiver oil, frankincense oil, galbanum oil, labdanum oil and lavandin oil.

The composition of the present disclosure may also contain cosmetically acceptable additives or adjuvants as well as cosmetic or dermatologic active agents. Representative additives and adjuvants include, for example, water-soluble or water-miscible solvents or co-solvents, dispersion enhancing agents, moisturizers, colorants, fillers, antioxidants (e.g., EDTA, BHT, tocopherol), essential oils, fragrances, dyes, neutralizing or pH-adjusting agents (e.g., citric acid, triethylamine (TEA) and sodium hydroxide), conditioning or softening agents (e.g., panthenol and allantoinin) and extracts such as botanical extracts. Additives and adjuvants may be present in the compositions in amounts generally ranging from about 0.01% to about 10% by weight. Examples of cosmetic active agents or dermatological active agents include sunscreen agents (e.g., inorganic sunscreen agent, such as titanium dioxide and zinc oxide and organic sunscreen agents, such as octocrylene, ethylhexyl methoxycinnamate, and avobenzone), free-radical scavengers, keratolytic agents, vitamins (e.g., Vitamin E and derivatives thereof), anti-elastase and anti-collagenase agents, peptides, fatty acid derivatives, steroids, trace elements, extracts of algae and of planktons, enzymes and coenzymes, flavonoids and ceramides, hydroxy acids and mixtures thereof, and enhancing agents. These ingredients may be soluble or dispersible in whatever phase or phases is/are present in the cosmetic composition (i.e., aqueous and/or fatty (oil) phase).

### SPF and UV-A and UV-B, and Whiteness

SPF (sun protection factor) is a measure of how effective sunscreen compositions will be able to provide protection from ultraviolet B (UV-B) radiation. The chief cause of reddening and sunburn, UV-B rays tend to damage the epidermis, skin's outer layers. The in vitro SPF a can be determined using methods known in the art. Nonetheless, a preferable method for determining in vitro SPF is the method described in Fageon, L. et al. Int. J. Cosmetic Sci., 2009, 405-17, which is incorporated herein by reference in its entirety.

UV-Filters, active in UV-A and/or UV-B regions, used for the present disclosure can be water-soluble, fat-soluble or insoluble in commonly used cosmetic solvents. UV-A filter comprises groups of compounds which absorb light predominantly in the range of wavelengths 400 nm to 320 nm (UV-A) and UV-B filter comprise groups of compounds which absorb light predominantly in the range of wavelengths 400 nm to 320 nm 320 nm to 280 nm (UV-B).

The sunscreen compositions of the instant disclosure may include an individual mineral UV filtering agents sun filters UV filter or a combination of mineral UV filtering agents sun filters UV filter. For example, sunscreen compositions may include two, three, four, five, or more UV filters. In some cases, the one or more UV filters is in an amount of from about 0.1%, 0.2%, 0.3%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5% to about 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, or 40 by weight based on the total weight of the composition.

The whiteness of a sunscreen composition can be determined by applying the sunscreen composition in a consistent manner to a substrate of choice and comparing the whiteness of the deposit visually using a panel of participants or colorimetrically using a suitable spectrophotometer. An example of method of application may the same manner of application described in Fageon et al. An example of a substrate may include a glass microscope slide, polymethylmethacrylate (PMMA) plate, or directly on skin, most preferably the volor forearm.

### Methods

The instant disclosure relates to methods for protecting a keratinous substrate, especially the skin and/or hair, from UV radiation, to methods of absorbing UV light, and to methods for preventing sunburn. Such methods comprise application of a sunscreen composition to a keratinous substrate, such as the skin and/or hair, and subjecting the keratinous substrate to ultraviolet radiation.

More exhaustive but non-limiting lists of components useful in the hair care compositions disclosed herein are presented below.

Implementation of the present disclosure is provided by way of the following examples. The examples serve to illustrate the technology without being limiting in nature.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular.

The expression “at least one” means one or more and thus includes individual components as well as mixtures/combinations.

All ranges and values disclosed herein are inclusive and combinable. For examples, any value or point described herein that falls within a range described herein can serve as a minimum or maximum value to derive a sub-range, etc.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### EXAMPLES

The following Examples are provided for illustrative purposes only, and are not intended to be limiting.

### Example 1: Inventive Example

Inventive Composition was prepared and is presented in Table 1, below.

TABLE 1

| | Inventive | Inventive |
|---|---|---|
| INCI US Name | Example 1 | Example 2 |
| Silica Silylate | 0.5 | 0.4 |
| Allantoin | 0.5 | 0.5 |
| Emulsifier | 8.8 | 7.6 |
| Dimethicone Crosspolymer | | |
| Film former | 0.8 | 0.8 |
| Acrylate Copolymer | | |
| Mineral UV filtering agents Filter | 9 | 13 |
| First silicone | 27.1 | 27.1 |
| 5 cst dimethicone | | |
| Second silicone | 51 | 44.4 |
| Dimethicone | | |

In making the formulations in the above tables, the following procedure was used.
1. First Silicone, allantoin and mineral UV filtering agents filter were added to steel beaker. Then, ran under Silverson at 5000 RPM for 15-minutes or until fully dispersed (no large particles). In between mixing, the sides of beaker were wiped by using a spatula.
2. The above phase was transferred into main kettle (ESCO), if present in composition, and the phase was then heated to 75 C. The phase was mixed using scraper at 30% and homogenizer at low 10% (without splashing).
3. The dimethicone crosspolymer and second silicone were added and removed from the heating jacket. The mixing was continued (50% scraper and homogenizer at 20%).
4. Once homogeneous a film former was added and mixing was continued (50% scraper and homogenizer at 20%).
5. Once homogeneous the silica silylate was added using a funnel (cover with plastic wrap to avoid particles released into air) while continuing to mix.
6. To ensure all silica silylate was mixed, spatula was used to wipe kettle and scraper.

### Example 2: Comparative Examples with and without Mineral UV Filtering Agents Filters

TABLE 2

| | | Inventive | Comp. | Comp. |
|---|---|---|---|---|
| INCI US Name | | Example 1 | 1 | 2 |
| Silica Silylate | Silica aerogel | 0.5 | 1 | 0.5 |
| Allantoin | Skin Protectant | 0.5 | 0.5 | 0.5 |
| Emulsifier | Blurring effect | 8.8 | 10.8 | 8.5 |
| Dimethicone | associate with | | | |
| Crosspolymer | silica aerogel | | | |
| Film former | | 0.8 | 0.8 | 0.8 |
| Acrylate Copolymer | | | | |
| Mineral UV filtering | Sun Filter | 9 | 0 | 9 |
| agents Filter | | | | |
| Fatty substance | Solid fatty | 0 | 2 | 2 |
| Helianthus Annuus | substance | | | |
| (Sunflower) Seed Oil | | | | |
| Unsaponifiables | | | | |
| Helianthus Annuus Seed | | | | |
| Oil Unsaponifiables | | | | |
| (SOLINE) | | | | |
| First silicone | Silicone Oil | 27.1 | 21.5 | 27.1 |
| 5 cst dimethicone | | | | |
| Second silicone | Silicone oil | 51 | 62.4 | 49.3 |
| Dimethicone | | | | |
| Stability | | YES | YES | NO |

The Inventive Example 1 and Comparative Examples 1 and 2 were prepared following the procedure described above.

The Inventive Example 1 was a blend of high concentration of silicones with dimethicone crosspolymer that created a barrier in the skin and results in a blur finish. In the Inventive Example 1, a low viscosity dimethicone (5 cSt) was used and no solid fatty substance was present. After the application of the Inventive Example 1 on the skin, it was observed that the skin was smoother and well moisturized. It was also observed that this application reduced the irritation of the skin and reduce the redness due to irritation. A significant blurring effect was observed. Furthermore, the formula was stable.

In the case of Comparative Example 1, there was no sun filters, but it did contain a solid fatty substance known as Helianthus annuus (sunflower) seed oil unsaponifiables. It was observed that the Comparative Example 1 was stable.

However, when sun filters were added to Comp. Ex. 1 and became the formula labelled Comp. Ex.2, it was observed that the formula was not stable and showed some separation.

It was then concluded that surprisingly the combination of mineral UV filtering agents sun filters with Helianthus annuus (sunflower) seed oil unsaponifiables resulted in an unstable formula. Indeed, when mineral UV filtering agents sun filters are combined with Helianthus annuus (sunflower) seed oil unsaponifiables, the formula is unstable.

What is claimed:

1. An anhydrous mineral sunscreen cosmetic composition comprising:
a) From about 0.1% to about 2% by weight of one silica silylate;
b) From about 0.1% to about 5% by weight of allantoin;
c) From about 1% to about 12% by weight of one at least one emulsifier;
d) From about 0.1 to about 5% by weight of at least one film former;
e) From about 1% to about 25% by weight of one or more mineral UV filtering agents;
f) From about 20% to about 30% by weight of at least a first silicone having a viscosity from about between 2 cst to about 70 cst; and
g) From about 40% to about 95% by weight of at least a second silicone;
wherein all weight percentages are based on the total weight of the cosmetic composition.

2. The anhydrous cosmetic composition of claim 1, wherein the silica silylate is silica silylate particles and especially aerogel particles of hydrophobic silica surface-modified with trimethylsilyl groups.

3. The anhydrous cosmetic composition of claim 2, wherein the silica silylate is present in an amount from about 0.5% to about 1.5% by weight based on the total weight of the composition.

4. The anhydrous cosmetic composition of claim 1, wherein the allantoin is present in an amount from about 0.1% to about 4% by weight based on the total weight of the composition.

5. The anhydrous cosmetic composition of claim 4, wherein the allantoin is present in an amount from about 0.4% to about 3% by weight based on the total weight of the composition.

6. The anhydrous cosmetic composition of claim 1, wherein the one at least one emulsifier comprises an organosiloxane emulsifier.

7. The anhydrous cosmetic composition of claim 6, wherein the organosiloxane emulsifier is a crosslinked organosiloxane emulsifier selected from the group consisting of dimethicone crosspolymer, dimethicone/dimethicone crosspolymer, dimethicone/dimethicone PEG/PPG 15 crosspolymer, dimethicone PEG-10 crosspolymer, dimethicone PEG-10/15 crosspolymer, dimethicone PEG-15 crosspolymer, dimethicone polyglycerin-3 crosspolymer, dimethicone PPG-20 crosspolymer, dimethiconol/methylsilanol/silicate crosspolymer; dimethiconol/silicate crosspolymer, lauryl dimethicone PEG-15 crosspolymer, lauryl dimethicone polyglycerin-3 crosspolymer, PEG-8 dimethicone polysorbate-20 crosspolymer, PEG-10 dimethicone/vinyl dimethicone crosspolymer, PEG-10 lauryl dimethicone crosspolymer, PEG-15/lauryl dimethicone crosspolymer, PEG-15 laurylpolydimethylsiloxyethyl crosspolymer.

8. The anhydrous cosmetic composition of claim 1, wherein at least one film former is selected from the group consisted of acrylates/dimethicone crosspolymer, Trimethylsiloxysilicate (and) polypropylsilsesquioxane, Acrylates/Polytrimethylsiloxymethacrylate Copolymer, Dimethicone (and) Acrylates/dimethicone copolymer, Polypropylsilsesquioxane, Trimethylsiloxysilicate), and a mixture thereof.

9. The anhydrous cosmetic composition of claim 1, wherein the one or more mineral UV filtering agents comprises titanium dioxide, zinc oxide, iron oxides, cerium oxides, zirconium oxides, alumina, aluminum hydroxide, or a mixture thereof.

10. The anhydrous cosmetic composition of claim 9 comprising titanium dioxide, zinc oxide, or a mixture thereof.

11. The anhydrous cosmetic composition of claim 1, wherein the one or more mineral UV filtering agents are present in an amount from about 3% to about 20% by weight based on the total weight of the composition.

12. The anhydrous cosmetic composition of claim 1, wherein the at least first silicone has a viscosity from between about 4 cst to about 65 cst by weight based on the total weight of the composition.

13. The anhydrous cosmetic composition of claim 1, wherein the at least second silicone has a viscosity of at least about 100 cSt.

14. The anhydrous cosmetic composition of claim 13, wherein the at least second silicone comprises dimethicone.

15. The anhydrous cosmetic composition of claim 1, wherein the at least second silicone is present from about 43% to about 90% by weight based on the total weight of the composition.

16. The anhydrous cosmetic composition of claim 1, further comprising:
h) One or more active agents.

17. The cosmetic composition of claim 16, wherein the one or more active agents are selected adenosine, 2-[4-(2-hydroxyethyl)piperazin-1-yl]ethanesulfonic acid (HEPES), hyaluronic acid, lanolin, citric acid, malic acid, lactic acid, tartaric acid, salicylic acid, vitamin C, a vitamin, a retinoid, retinal, retinoic acid, a carotenoid, an amino acid, a protein, an enzyme, a coenzyme, and mixtures thereof.

18. An anhydrous mineral sunscreen cosmetic composition comprising:
a) From about 0.1% to about 2% by weight of one silica silylate;
b) From about 0.1% to about 5% by weight of allantoin;
c) From about 1% to about 12% by weight of dimethicone crosspolymer;
d) From about 0.1 to about 2% by weight of at least one film former selected from the group consisted of acrylates/dimethicone crosspolymer, Trimethylsiloxysilicate (and) polypropylsilsesquioxane, Acrylates/Polytrimethylsiloxymethacrylate Copolymer, Dimethicone (and) Acrylates/dimethicone copolymer, Polypropylsilsesquioxane and mixture thereof.
e) From about 3% to about 20% by weight of mineral UV filtering agents sun filters by weight based on the total weight of the composition;
f) From about 20% to about 30% by weight of at least a first silicone having a viscosity from about between 2 cst to about 70 cst; and
g) From about 40% to about 95% by weight of at least a second silicone;
wherein all weight percentages are based on the total weight of the cosmetic composition.

19. The anhydrous mineral sunscreen cosmetic composition of claim 1, wherein the composition exhibits an SPF of at least 15.

20. The anhydrous mineral sunscreen cosmetic composition of claim 1, wherein the composition exhibits an SPF of at least 20.

21. The anhydrous mineral sunscreen cosmetic composition of claim 1, wherein the composition exhibits an SPF of at least 30.

22. A method for protecting skin from UV radiation comprising applying an effective amount of the sunscreen composition of claim 1 to the skin.
